(19) United States
(12) Patent Application Publication
(10) Pub. No.: US 2005/0232946 A1
(43) Pub. Date: Oct. 20, 2005

(54) Vaccine against microbial pathogens

(76) Inventor: Camilo Anthony Leo Selwyn Colaco, Cambridge (GB)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(21) Appl. No.: 10/363,454

(22) PCT Filed: Sep. 4, 2001
(86) PCT No.: PCT/GB01/03964

§ 371 Date: Jun. 10, 2003

(30) Foreign Application Priority Data
Sep. 4, 2000 (GB) 0021757.0

(51) Int. Cl. A61K 39/12; A61K 39/02

(52) U.S. Cl. 424/199.1; 424/200.1

(57) ABSTRACT

The present invention provides a vaccine comprising a microbial pathogen, wherein the microbial pathogen is subjected to a stress inducing stimuli. The stress inducing stimuli can be heat or osmotic stress, through preferably the microbial pathogen is genetically modified such that at least one repressor gene for a heat shock protein gene is inactivated, thus allowing the constitutive expression of heat shock proteins. In particular the use of heat shock protein repressor mutant bacteria is shown to be effective for inducing immunity when comprised within vaccines of the present invention. The present invention further provides a method for producing a vaccine comprising stressed induced microbial pathogens and further the use of the heat shock protein repressor deletion mutant microbes as vaccine vectors which can be additionally allow the expression of heterologous antigen fragments.

The present invention relates to a vaccine and a method for producing a vaccine. More specifically, there is provided a vaccine comprising a microbial pathogen and a method of producing the same.

### BACKGROUND OF THE INVENTION

An important component of any human immune response is the presentation of antigens to T cells by antigen presenting cells (APCs) such as macrophages, B cells or dendritic cells. Peptide fragments of foreign antigens are presented on the surface of the macrophage in combination with major histocompatibility complex (MHC) molecules, in association with helper molecules, such as CD4 and CD8 molecules. Such antigenic peptide fragments presented in this way are recognised by the T cell receptor of T cells. The interaction of the antigenic peptide fragments with the T cell receptor results in antigen-specific T cell proliferation, and secretion of lymphokines by the T-cells. The nature of the antigenic peptide fragment presented by the APCs is critical in establishing immunity.

Heat shock proteins (hsps) form a family of highly conserved proteins that are widely distributed throughout the plant and animal kingdoms. On the basis of their molecular weights, hsps are grouped into six different families: small (hsp 20-30 kDa); hsp40; hsp60; hsp70; hsp90; and hsp100. Although hsps were originally identified in cells subjected to heat stress, they have been found to be associated with many other forms of stress, such as infections, and are thus also commonly referred to as stress proteins (SPs).

Members of the mammalian hsp90 family include cytosolic hsp90 (hsp83) and the endoplasmic reticulum counterparts hsp90 (hsp83), hsp87, Grp94 (Erp99) and gp97, see for example Gething et al. (1992) Nature 355:33-45. Members of the hsp70 family include cytosolic hsp70 (p73) and hsp70 (p72), the endoplasmic reticulum counterpart BiP (Grp78), and the mitochondrial counterpart hsp70 (Grp75). Members of the mammalian hsp60 family have only been identified in the mitochondria.

Stress proteins are ubiquitously expressed within cells. One of the roles of stress proteins is to chaperone peptides from one cellular compartment to another and to present peptides to the MHC molecules for cell surface presentation to the immune system. In the case of diseased cells, stress proteins also chaperone viral or tumour-associated peptides to the cell-surface, see Li and Sirivastave (1994) Behring Inst. Mitt, 94: 37-47 and Suzue et al. (1997) Proc. Natl. Acad. Sci. USA 94: 13146-51.

The chaperone function of stress proteins is accomplished through the formation of complexes between stress proteins and antigenic peptide fragments, and between stress proteins and viral or tumour-associated peptide fragments, in an ATP dependent reaction. The peptide fragments complexed with the stress proteins form antigenic complexes (hspCs) which are captured by APCs to provide antigenic peptide fragments.

The complex association of stress proteins with peptide fragments has also been observed in normal tissues and is not therefore a tumour-specific phenomenon, see Srivastava (1994) Experimentia 50: 1054-60. It is thought that the immunogenicity of the members of the hsp70 family, including grp96, reflects their normal role in the ‘presentosome’, the postulated intracellular organelle functioning in the loading of MHC Class I molecules required for their cell surface expression. This step is essential for their MHC-restricted recognition by antigen-specific T-cells, see Srivastava et al. (1998) Immunity, Singh-Jasuja et al. J. Exp. Med (2000) 191, 1957.

Until recently, it has not been proposed to use pathogen-derived endogenous hsp-peptide complexes (hspCs) as vaccines, despite the fact that hsps from pathogens have been used extensively as antigens and adjuvants. PCT Application No GB00/03228 discloses the use of pathogen-derived endogeneous hspCs and in particular stress induced hspCs are shown to give good protective immunity in vaccinated animals.

Members of the microbial hsp family include the Dna J and Dna K families and the Gro-EL and Gro-ES families. In prokaryotic microbes it appears that these families are encoded in operons, with the initial gene in the operon being a control gene which suppresses the expression of the hsp genes contained within the operon.

In Streptomyces and Helicobacter, expression of the hspR gene suppresses the expression of Dna J and Dna K. Deletion of the hspR gene therefore results in a genetically modified microbe that constitutively expresses hsps, see Bucca et al. (1997) Mol. Microbiol 15:633-45. Homologous operons have been identified in a number of recently sequenced microbes, including other strains of Streptomyces and Mycobacterium tuberculosis and the commonly used related vaccine strain BCG.

Other repressor genes may also control the expression of members of the hsp gene family and that these may also be genetically engineered to provide modified microbes that constitutively expresses hspCs that may be utilised for the production of vaccines and vaccine vectors. These include, but are not limited to the transcriptional control genes sigma and rho and the stress-gene regulatory protein genes MerR and HmrR. It will also be appreciated that the modified microbes containing the constitutive hspCs may be used directly as vaccines or as a source for the isolation of the hspCs. Furthermore the expression of heterologous gene(s) from other pathogen(s) in these microbes will enable their use as vaccine vectors for the simplified production of hspC-based vaccines for these pathogens.

Whilst looking at the use of heat-induced endogeneous microbial hsp-peptide complexes, the extracted hsp-peptide complexes were compared to the use of the stressed-induced microbe itself as a vaccine. Surprisingly, the use of the microbe as a vaccine gave significantly better immunity in vaccinated animals than the use of the isolated hsp-peptide complexes. Similar results were obtained using genetically modified microbes that constitutively produced hsps, indicating that hspCs were formed in situ with endogenous microbial polypeptides, including heterogenous genes expressed as recombinant proteins in the microbe. The genetically modified microbes could also be used as an efficient source for the isolation of hspCs for use in subunit and multi-subunit vaccines.

### SUMMARY OF THE INVENTION

According to the present invention, there is provided a vaccine comprising a microbial pathogen as an immunogenic determinant, wherein the microbial pathogen has been subjected to a stress inducing stimuli.

Preferably the stress inducing stimuli results in the expression of heat shock proteins by the microbe.

Preferably the stress inducing stimuli is heat or osmotic shock.

More preferably the stress inducing stimuli is the genetic modification of the microbial pathogen such that at least one repressor gene for a heat shock protein gene is inactivated, thereby allowing the expression of a heat shock protein gene.

Preferably the microbial pathogen is any pathogen which is capable of inducing an infectious disease.

Preferably the microbial pathogen is a bacteria, protozoa, fungi or a parasitic organism.

Preferably the genetic modification results in the inactivation of the hspR repressor gene.

Alternatively the genetic modification results in the inactivation of the stress gene regulatory protein genes MerR or HmrR repressor genes, or the transcriptional control genes sigma and rho.

Preferably the microbial pathogen is selected from the group consisting of Mycobacteria, Salmonella, Vibrio, Streptomyces, Helicobacter, Lactococcus and Listeria.

Preferably the microbial pathogen is attenuated.

Preferably the vaccine further comprises an adjuvant.

Preferably the adjuvant is selected from the group consisting of; Freund's complete adjuvant, Freund's incomplete adjuvant, Quil A, Detox, ISCOMs and squalene.

Preferably the vaccine is suitable for administration by injection.

Alternatively the vaccine is suitable for oral administration.

Preferably the vaccine is suitable for delivery by means of a needle-less delivery format.

A further aspect of the present invention provides a method of vaccinating an animal, characterised in that said method comprises administering a pharmaceutically acceptable quantity of a vaccine composition according to the present invention sufficient to elicit an immune response in the animal.

Preferably the vaccine is administered as a prophylactic vaccine.

Alternatively the vaccine is administered as a therapeutic vaccine.

Preferably the vaccine composition is administered by injection.

Alternatively the vaccine composition is administered by needle-less delivery.

Alternatively the vaccine composition is administered transdermally, by pulmonary delivery or orally.

A yet further aspect of the present invention provides a method of producing a vaccine composition, comprising an immunogenic determinant, characterised in that said method comprises the steps of; subjecting microbial pathogens to stress inducing stimuli; and using the stressed microbe in the preparation of the vaccine composition as said imunogenic determinant.

Preferably the stress inducing stimuli is heat or osmotic shock.

More preferably the stress inducing stimuli is the inactivation of a gene which represses the expression of heat shock genes.

Preferably the repressed gene is the hspR gene.

Alternatively the repressed gene is the stress gene regulatory protein gene MerR or HmrR, or the transcriptional control genes sigma and rho.

Preferably the microbial pathogen cells are killed prior to use in said vaccine.

Alternatively the microbial pathogen cells are dried prior to use in said vaccine.

Preferably the vaccine composition is an aqueous composition.

Alternatively the vaccine composition is a dry composition or in a lyophilised composition.

The present invention further provides the use of a pathogenic microbe which has been genetically modified such that at least one repressor gene for a heat shock protein is inactivated, thereby allowing constitutive expression of heat shock proteins, as a vaccine vector which further allows the expression of a heterologous antigen.

Preferably the pathogenic microbe is a prokaryotic organism, which is suitable for use as a vaccine vector.

Preferably the prokaryotic organism is BCG and the heterologous antigen fragment is the tetanus toxoid fragment C.

Alternatively the prokaryotic organism is Salmonella or Lactococcus, such that the vaccine can be administered orally.

The present invention further provides the use of a pathogenic microbe which has been genetically modified such that at least one repressor gene for a heat shock protein is inactivated, thereby allowing constitutive expression of heat shock proteins, as a source of heat shock protein-peptide complexes for use in subunit and multi-subunit vaccines.

By “microbial pathogen” it is meant any pathogen capable of inducing an infectious disease in an animal, including particularly bacterial, protozoan, fungal or parasitic organisms.

By “stress-inducing stimuli” it is meant any stimuli that induces the production of SPs and in particular hsps in microbial pathogens, including heat or osmotic stress. Such stress-inducing stimuli also includes genetic changes designed to render constitutive the expression of stress proteins and in particular hsps in microbial pathogens. These genetic changes include the inactivation of repressor (suppressor) genes that function in the suppression of stress proteins and in particular the suppression of the genes which express heat shock proteins. This includes in particular the inactivation of the hspR suppressor gene, the stress gene regulatory protein genes MerR and HmrR, or the transcriptional control genes sigma and rho.

It will be appreciated that such genetic changes are readily achieved by molecular genetic means and include insertional mutagenesis using phage or transposon vectors, see for example Bucca et al. Mol. Microbiol. (1997) 17:633. It will also be appreciated that the presence of the genes for the operons, suppressor genes and SPs and hsps in microbial pathogens can be simply established by recombinant DNA techniques, see Current Techniques in Molecular Biology, (1999) Wiley Press.

Thus it should be appreciated that any microbe expressing these genes can be genetically modified to provide a vaccine according to this invention.

This includes existing microbial organisms used as live or killed vaccines, such as Mycobacteria, Salmonella, Vibrio and Listeria and in particular attenuated varieties of these pathogens. In particular, Mycobacteria mutants, which have been genetically modified to delete the heat shock protein repressor gene could be used in a vaccine against tuberculosis, while Salmonella mutants which have been genetically modified to delete the heat shock protein repressor genes could be used in a vaccine against typhoid. The use of such organisms as vaccine vectors for the expression of heterologous genes is further provided by the present invention.

The term “vaccine” is used herein to denote to any composition containing an immunogenic determinant which stimulates the immune system such that it can better respond to subsequent infections. It will be appreciated that a vaccine usually contains an immunogenic determinant and an adjuvant, the adjuvant serving to non-specifically enhance the immune response to that immunogenic determinant.

Suitable adjuvants are readily apparent to the person skilled in the art, and include; Freund's complete adjuvant, Freund's incomplete adjuvant, Quil A, Detox, ISCOMs or squalene.

However, it will be appreciated that the vaccine of the present invention may also be effective without an adjuvant.

For the non-genetic induction of SPs, the optimum conditions for inducing the SPs can readily be determined by simple trial and error with the effect of a change of stimuli being assessed using conventional techniques, such as in vivo testing on animals, or by other techniques, for example those described in ‘Current Protocols in Immunology’, Wiley Interscience, 1997. Other such conditions are described in PCT Application No GB00/03228 and citations referred to therein.

The invention also provides a method for exposing an animal to a vaccine of the invention by administering a pharmaceutically acceptable quantity of the vaccine of the invention, optionally in combination with an adjuvant, sufficient to elicit an immune response in the animal.

The animal is typically a human. However, the invention can also be applied to the treatment of other mammals such as horses, cattle, goats, sheep or swine, and to the treatment of birds, notably poultry such as chicken or turkeys. Preferably the microbial pathogen selected for use in a particular vaccine of the present invention causes disease or infection in the species of animal to which the vaccine is administered to, or a closely related species. The vaccines of this invention may be used as both prophylactic or therapeutic vaccines though it will be appreciated that they will be particularly useful as prophylactic vaccines due to their economy of production.

The vaccine compositions of the present invention may be administered by any suitable means, such as orally, by inhalation, transdermally or by injection and in any suitable carrier medium. However, it is preferred to administer the vaccine as an aqueous composition by injection using any suitable needle or needle-less technique.

It will be appreciated that the vaccine of the invention may be applied as an initial treatment followed by one or more subsequent “booster” treatments at the same or a different dosage rate at an interval of from 1 to 26 weeks between each treatment to provide prolonged immunisation against the pathogen.

The present invention will now be described, by way of example, with reference to the accompanying figures, wherein;

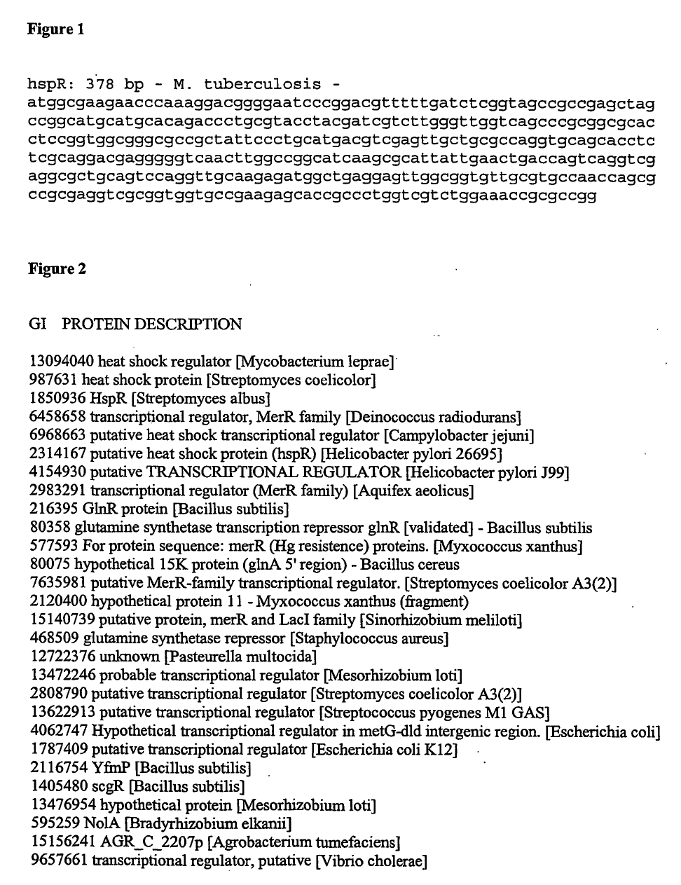

FIG. 1 shows the nucleotide sequence of the hspR gene of M. tuberculosis, and

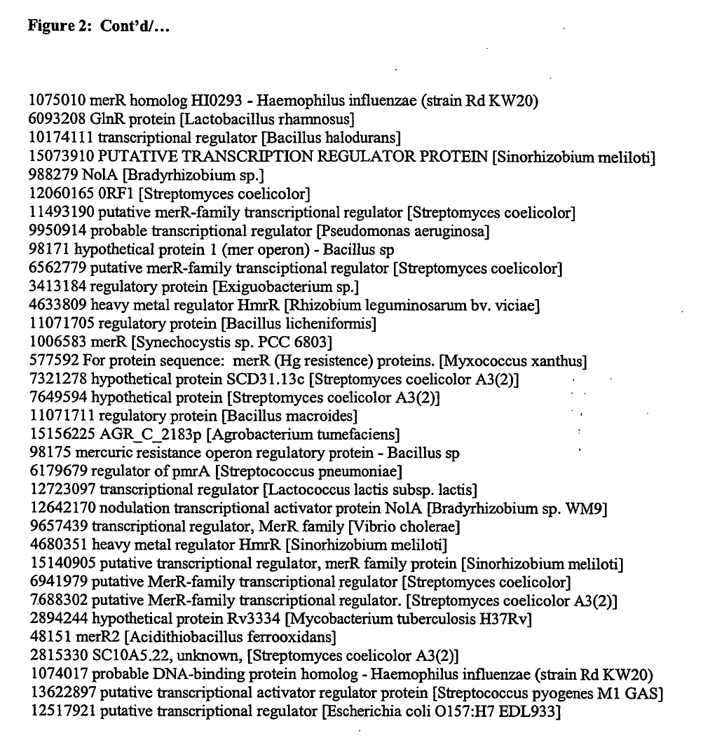

FIG. 2 shows a list of identified suppressor genes which have homology to hspR.

### EXAMPLE 1

### Preparation of Heat-Induced Microbes

M. vaccae strain NCTC11659 was grown to saturation in Sauton's media, diluted into fresh media and grown overnight to give a log phase culture which was then heat-shocked at 42° C. for 3 hours or at 39° C. for 5 hours and cultured overnight. The cells were then washed in media, followed by a wash in saline and either lyophilised in individual vaccine aliquots or used directly for the immunisation of test animals. For comparison isolated endogeneous SP-peptide complexes were prepared as described in PCT Application No GB00/03228. Essentially, washed stress-induced cells are then re-suspended in homogenisation buffer such as PBS with 0.5% Tween and cells are then disrupted by freeze-thaw cycles or using cell disruptor (e.g. bead beater, French press). The cell lysate is then treated by centrifugation, typically 3-5000 g for 5 minutes, to remove the nuclei and cell debris, followed by a high speed centrifugation step, typically 100,000 g for 15-30 minutes. The supernatant thus obtained is processed to give an SP/antigenic peptide fragment complex suitable for use in a vaccine. This can be done simply by ammonium sulphate precipitation which uses a 20-70% ammonium sulphate cut. Specifically, 20% (w/w) ammonium sulphate is added at 4° C., the precipitate is discarded, followed by the addition of more ammonium sulphate to bring the concentration to 70% w/w. The protein precipitate is harvested by centrifugation and then dialysed into an appropriate physiological, injectable buffer, such as saline, to remove the ammonium sulphate before use. The SP complexes may be used at any suitable concentration to provide the immunogenic determinant in the vaccine composition.

It is preferred that the amount of the induced stress protein-peptide complex is in the range of 10-600 μg, more preferably 10-100 μg, and most preferably 25 μg per kg of animal body weight.

In order to determine the immunogenicity of the SP complexes, T cell proliferation assays may be used. Suitable assays include the mixed-lymphocyte reaction (MLR), assayed by tritiated thymidine uptake, and cytotoxicity assays to determine the release of ⁵¹Cr from target cells, see ‘Current Protocols in Immunology’, Wiley Interscience, 1997. For Mycobacteria, MLRs can also be assayed for the induction of cytokine production such as the production of interferon gamma using the commercial kit (CSL Ltd). Alternatively, antibody production may be examined, using standard immunoassays or plaque-lysis assays, or assessed by intrauterine protection of a foetus, see ‘Current Protocols in Immunology’.

Mice were immunised with either heat-stressed organisms or the endogenous SP-peptide complexes isolated from the heat stressed organisms in phosphate buffered saline without any added adjuvant in either the primary or booster vaccinations.

Immunisation with whole stress-induced organisms and in particular lyophilised organisms gave significantly better immunity than that induced by isolated endogeneous SP-peptide complexes including increased IFN-gamma and antibody production.

### EXAMPLE 2

### Preparation and Use of Constitutive hsp Mutants

Constitutive hsp producing microbes can be constructed through the knockout of transcriptional regulator suppressor genes such as hspR, MerR (mercuric resistance operon regulatory protein), HmrR (heavy metal regulatory protein) and their homologues or the transcriptional control genes rho and sigma. Such genes are readily identified by screening of genome sequence databases with typical test sequences. An example of such a typical test sequence is the hspR gene of M. tuberculosis (shown in FIG. 1). An example of a list of identified suppressors which have homology to the hspR gene is shown in FIG. 2.

M. bovis strains constitutively producing hspCs were constructed by genetically engineering the M. bovis strain BCG to yield deletion mutants for the hspR gene. The hspR gene was deleted by homologous recombination using a suicide vector carrying a large fragment of the hspR gene and a kanamycin selection marker. The hspR gene fragment was cloned by PCR from BCG genomic DNA using primers derived from the M. tuberculosis hspR sequence shown in FIG. 1. Such an approach should be widely applicable to the production of similar mutants from any prokaryote carrying the relevant suppressor gene.

BCG hspR deletion mutants were used to produce hspCs and the protein yields obtained from these were comparable to those obtained from heat shocked wild type strains. Such an approach should again be widely applicable to the production of hspCs from any prokaryote carrying the relevant suppressor gene. These would provide a valuable resource for the manufacture of hspC-based vaccines. Thus hspCs produced from BCG hspR deletion mutants induced protective immunity against aerosol challenge by M. tuberculosis comparable to that induced by hspCs obtained from heat shocked wild type BCG strains.

BCG hspR deletion mutants can be also be used as vaccine vectors to express heterologous antigens. For example, mice immunised with a BCG hspR deletion mutant expressing the tetanus toxoid (TT) fragment C showed the production of anti-TT antibodies as detected by Western blotting. Such an approach should be widely applicable to the production of similar vectors for the expression of heterologous vaccine antigens using mutants from any prokaryote suitable for use as a vaccine vector. For example the use of Salmonella or Lactococcus mutants would enable the production of vectors targeted for mucosal delivery of vaccines. Vaccines developed on this principle would be particularly advantageous in that they could be administered orally.

1. A vaccine comprising a microbial pathogen as an immunogenic determinant, wherein the microbial pathogen has been subjected to a stress inducing stimuli.

2. A vaccine as claimed in claim 1 wherein the stress inducing stimuli results in the expression of heat shock proteins by the microbe.

3. A vaccine as claimed in claim 1 or claim 2, wherein the stress inducing stimuli is heat or osmotic shock.

4. A vaccine as claimed in claims 1 to 3, wherein the stress inducing stimuli is the genetic modification of the microbial pathogen such that at least one repressor gene for a heat shock protein gene is inactivated, thereby allowing the expression of a heat shock protein gene.

5. A vaccine as claimed in any of the preceding claims, wherein the microbial pathogen is any pathogen which is capable of inducing an infectious disease.

6. A vaccine as claimed in any of the preceding claims, wherein the microbial pathogen is a bacteria, protozoa, fungi or a parasitic organism.

7. A vaccine as claimed in claims 4 to 6, wherein the genetic modification results in the inactivation of the hspR repressor gene.

8. A vaccine as claimed in claims 4 to 6, wherein the genetic modification results in the inactivation of the stress gene regulatory protein genes MerR or HmrR.

9. A vaccine as claimed in claims 4 to 6, wherein the genetic modification results in the inactivation of the transcription control genes rho or sigma.

10. A vaccine as claimed in any of the preceding claims wherein the microbial pathogen is selected from the group consisting of Mycobacteria, Salmonella, Vibrio, Listeria, Streptomyces, Helicobacter and Lactococcus.

11. A vaccine as claimed in any of the preceding claims wherein the microbial pathogen is attenuated.

12. A vaccine as claimed in any of the preceding claims, wherein the vaccine further comprises an adjuvant.

13. A vaccine as claimed in claim 12, wherein the adjuvant is selected from the group consisting of; Freund's complete adjuvant, Freund's incomplete adjuvant, Quil A, Detox, ISCOMs and squalene.

14. A vaccine as claimed in any of the preceding claims, wherein the vaccine is suitable for administration by injection.

15. A vaccine as claimed in claims 1 to 13 wherein the vaccine is suitable for oral administration.

16. A vaccine as claimed in claim 14, wherein the vaccine is suitable for delivery by means of a needle-less delivery format.

17. A method of vaccinating an animal, characterised in that said method comprises administering a pharmaceutically acceptable quantity of a vaccine composition as claimed in any of claims 1 to 13, sufficient to elicit an immune response in the animal.

18. A method as claimed in claim 17 wherein said vaccine is administered as a prophylactic vaccine.

19. A method as claimed in claim 17 wherein said vaccine is administered as a therapeutic vaccine.

20. A method as claimed in claims 17, wherein the vaccine composition is administered by injection.

21. A method as claimed in claims 17, wherein the vaccine composition is administered by needle-less delivery.

22. A method as claimed in claims 17, wherein the vaccine composition is administered transdermally.

23. A method as claimed in claims 17, wherein the vaccine composition is administered by pulmonary delivery.

24. A method as claimed in claim 17, wherein the vaccine composition is administered orally.

25. A method of producing a vaccine composition, comprising an immunogenic determinant, characterised in that said method comprises the steps of:
subjecting microbial pathogens to stress inducing stimuli; and
using the stressed microbe in the preparation of the vaccine composition as said imunogenic determinant.

26. A method as claimed in claim 24, characterised in that the stress inducing stimuli is heat or osmotic shock.

27. A method as claimed in claim 25, characterised in that the stress inducing stimuli is the inactivation of a gene which represses the expression of heat-shock genes.

28. A method as claimed in claim 25, characterised in that the repressor gene is the hspR gene.

29. A method as claimed in claim 25, characterised in that the repressor gene is MerR or HmrR.

30. A method as claimed in any one of the preceding claims, characterised in that the microbial pathogen cells are killed prior to use in said vaccine.

31. A method as claimed in any of the preceding claims, characterised in that the microbial pathogen cells are dried prior to use in said vaccine.

32. A vaccine composition as claimed in any one of claims 25 to 31, characterised in that the composition is an aqueous composition.

33. A vaccine composition as claimed in any one of claims 25 to 31, characterised in that the composition is a dry composition.

34. A vaccine composition as claimed in claims 25 to 31, characterised in that the composition is a lyophilised composition.

35. Use of a pathogenic microbe which has been genetically modified such that at least one repressor gene for a heat shock protein is inactivated, thereby allowing constitutive expression of heat shock proteins, as a vaccine vector which further allows the expression of a heterologous antigen.

36. Use of pathogenic microbe as claimed in claim 34 wherein the pathogenic microbe is a prokaryotic organism, which is suitable for use as a vaccine vector.

37. Use of pathogenic microbe as claimed in claim 35 wherein the prokaryotic organism is BCG and the heterologous antigen fragment is the tetanus toxoid fragment C.

38. Use of pathogenic microbe as claimed in claim 34 wherein the prokaryotic organism is Salmonella or Lactococcus, such that the vaccine can be administered orally.

39. Use of a pathogenic microbe which has been genetically modified such that at least one repressor gene for a heat shock protein is inactivated, thereby allowing constitutive expression of heat shock proteins, as a source of heat shock protein-peptide complexes for use in subunit and multi-subunit vaccines.


SEQUENCE LISTING (1 sequence)

SEQ ID NO: 1 - DNA, 378 bp, M. tuberculosis
atggcgaagaacccaaaggacggggaatcccggacgtttttgatctcggtagccgccgagctagccggcatgcatgcacagaccctgcgtacctacgatcgtcttgggttggtcagcccgcggcgcacctccggtggcgggcgccgctattccctgcatgacgtcgagttgctgcgccaggtgcagcacctctcgcaggacgagggggtcaacttggccggcatcaagcgcattattgaactgaccagtcaggtcgaggcgctgcagtccaggttgcaagagatggctgaggagttggcggtgttgcgtgccaaccagcgccgcgaggtcgcggtggtgccgaagagcaccgccctggtcgtctggaaaccgcgccgg